(19) United States
(12) Patent Application Publication
(10) Pub. No.: US 2023/0364544 A1
(43) Pub. Date: Nov. 16, 2023

(54) A PANEL FOR AN AIR CIRCULATION SYSTEM

(71) Applicants: Wayne Neville SEEGERS, Johannesburg (ZA); Richard Graham SEEGERS, Fourways (ZA); Alistair William SEEGERS, Honeydw (ZA)

(72) Inventor: Graham Neville SEEGERS, Randburg (ZA)

(21) Appl. No.: 18/027,908

(22) PCT Filed: Sep. 23, 2020
(86) PCT No.: PCT/IB2020/058866

§ 371 Date: Mar. 31, 2023

(51) Int. Cl. B01D 46/00; B01D 53/32

(52) U.S. Cl. CPC B01D 46/0032; B01D 53/32; B01D 2257/91

(57) ABSTRACT

A panel (100) is for an air circulation system and includes a foam layer (102) comprising open pore foam, a support structure (112, 114) configured to support the foam layer (102), and a graphite coating (104) provided on at least one side of the foam layer (102). The panel (100) has an ionizer (120) provided adjacent the foam layer (102), the ionizer (120) configured to ionize particles (122) thereby to apply a static charge to the foam layer (102) or the graphite coating (104). The graphite coating (104) layer is configured, in use, to attract and trap contaminants from air.

### FIELD OF INVENTION

This invention relates generally to air purification and specifically to a panel for an air circulation system, wherein the panel is not, in itself, a filter.

### BACKGROUND OF INVENTION

The Applicant is aware of existing air filters using graphite or graphene (see, for example, ZA Patent No. 2020/02355). However, a filter, by the Applicant's definition, includes a member or filter medium through which fluid (e.g., air) must be passed and which filters out contaminants from the air. The applicant desires to improve on this idea by providing an apparatus which can remove contaminants from the air, but which is not a filter.

### SUMMARY OF INVENTION

Accordingly, the invention provides a panel for an air circulation system, the panel including:
a foam layer comprising open pore foam;
a support structure configured to support the foam layer;
a graphite coating provided on at least one side of the foam layer; and
an ioniser provided adjacent the foam layer, the ioniser configured to ionise particles thereby to apply a static charge to the foam layer or the graphite coating,
wherein the graphite coating layer is configured, in use, to attract and trap contaminants from air.

The term “contaminants” may include pathogens, bacteria, viruses, microbes, etc.

The foam layer may be of dielectric-type foam. The foam may be of polyurethane. The foam layer may include a without charcoal filling. The foam layer may be 20-30 mm thick.

The support structure may include a support layer. The support layer may be provided parallel to the foam layer. The support layer may be provided on one or both sides of the foam layer. The support layer may be of a rigid polymer, e.g., plastic. The support layer may include a grid, mesh, or web of support structures. The support layer may resemble an eggcrate. The graphite coating may be provided between the foam layer and the support structure.

The support structure may include a frame provided around sides of the foam layer. The frame may have a square or rectangular footprint and a C-shaped cross-sectional profile. The frame may be of metal.

The graphite coating may be provided on both sides of the foam layer.

The graphite coating may include graphene. The graphite coating may include graphite powder. The graphite powder may be 7-9 microns in diameter. The graphite coating may include 50-80% graphite powder and may include 70% graphite powder.

The graphite coating may include an acid which may be phosphoric acid. The graphite coating may include 5-20% phosphoric acid and specifically may include 10% phosphoric acid.

The graphite coating may include a bonding agent. The graphite coating may include 10-30% bonding agent and specifically may include 20% bonding agent. The bonding agent may be acrylic- or polyurethane-based.

The graphite coating may be 1-2 mm thick. The graphite coating may be applied to the foam layer by spraying.

The panel may find application in systems or apparatus which cause or control airflow, e.g., HVAC (Heating, Ventilation, and Air-Conditioning) systems in buildings, vacuum cleaners, portable fans, etc. In one implementation, the panel may be installed in or adjacent an HVAC inlet or outlet. The panel maybe in, or close to, a ceiling of a building. The ceiling may be a suspended ceiling or a solid ceiling. The panel may be approx. 600 mm×400 mm or a size to fit the application.

The panel may include attachment formations for attaching the panel to a base structure, e.g., a ceiling. The attachment formations may be connected to the support structure. The attachments formations may be plugs (e.g., for solid ceilings) or clips (e.g., for suspended ceilings).

The ioniser may be an electronic ioniser. The ioniser may emit (1-6×1096) pcs/cm³ 8 kVA particles. The ioniser may be a 6-8 kV ioniser. The ioniser (or an outlet thereof) may be spaced approximately 5 mm from the foam layer. Where the graphite coating is on only one side of the foam layer, the ioniser may be placed on the other side. The ioniser may, in use, consume 2 W or less of power. The ioniser may be operational permanently or indefinitely.

The panel may include electrodes. The electrodes may be sandwiched between the support structure and the graphite coating. The electrodes may be 12-24 VAC or VDC. There may be two electrodes. The electrodes may be spaced apart and parallel. The electrodes may be brass or copper.

The invention extends to an air circulation system which includes the panel defined above which may be provided adjacent to or inline with an airflow path. The air circulation system may exclude an air filter.

The invention extends to a method of manufacturing the panel as described above, the method including:
mixing graphite powder with an acid and a bonding agent;
stirring the mixture to exfoliate it into amorphous particle fullerene graphene structures; and
applying the mixture to the foam layer.

The structures may be flakes or nanotubes.

The mixing may include the following ratios (by weight):
50-80% graphite powder, e.g., 70% graphite powder;
5-20% acid, e.g., 10% acid; and
10-40% bonding agent, e.g., 20% bonding agent.

The acid may be phosphoric acid. The bonding agent may be acrylic or polyurethane-type.

The stirring may be for 5-10 minutes. The stirring may be continuous.

The applying may include spraying. The spraying may be with an airless-type spray gun. The mixture may be sprayed to a thickness of 1-2 mm.

The method may include drying the panel in an oven or similar heat applicator. The drying may be at 60° C. The drying may be until the mixture is dry.

The method may include providing electrodes on the applied mixture.

The method may include attaching the support structure to the foam layer. The attaching may be before or after the applying of the mixture.

### BRIEF DESCRIPTION OF DRAWINGS

The invention will now be further described, by way of example, with reference to the accompanying diagrammatic drawings.

In the drawings:

FIG. 1 shows a three-dimensional view from a top of a panel, in accordance with the invention;

FIG. 2 shows a three-dimensional view from a bottom of the panel of FIG. 1;

FIG. 3 shows a top plan view of the panel of FIG. 1; and

FIG. 4 shows a cross-sectional view through section A-A of the panel of FIG. 1.

### DETAILED DESCRIPTION OF EXAMPLE EMBODIMENT

The following description of an example embodiment of the invention is provided as an enabling teaching of the invention. Those skilled in the relevant art will recognise that changes can be made to the example embodiment described, while still attaining the beneficial results of the present invention. It will also be apparent that some of the desired benefits of the present invention can be attained by selecting some of the features of the example embodiment without utilising other features. Accordingly, those skilled in the art will recognise that modifications and adaptations to the example embodiment are possible and can even be desirable in certain circumstances and are a part of the present invention. Thus, the following description of the example embodiment is provided as illustrative of the principles of the present invention and not a limitation thereof.

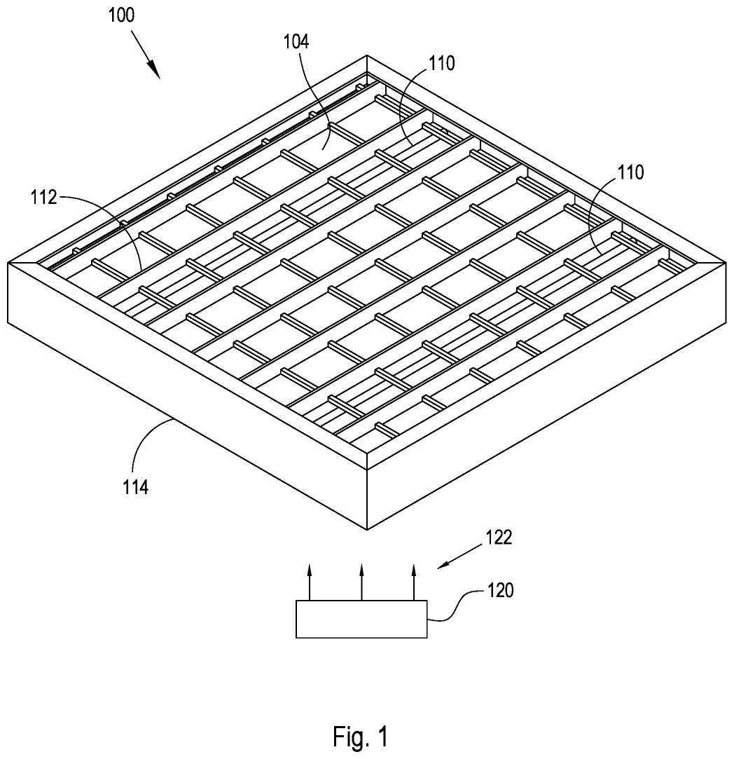

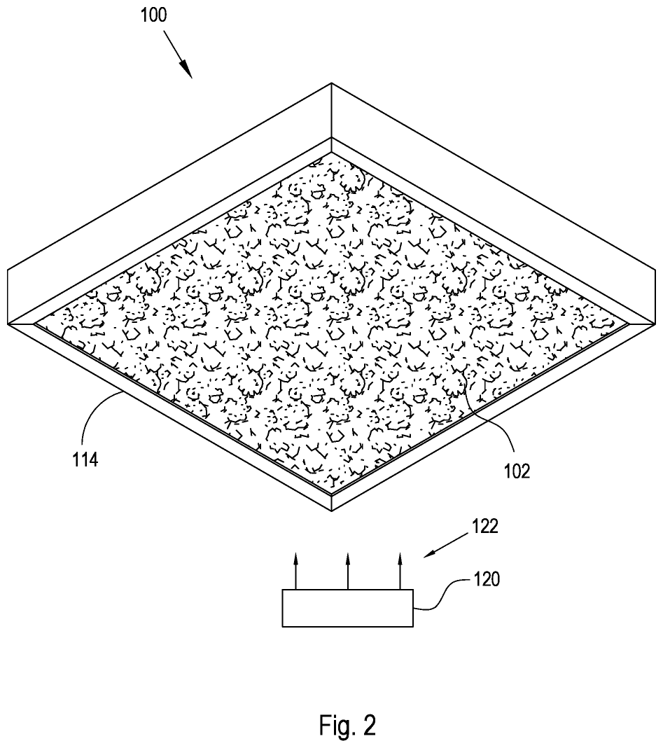

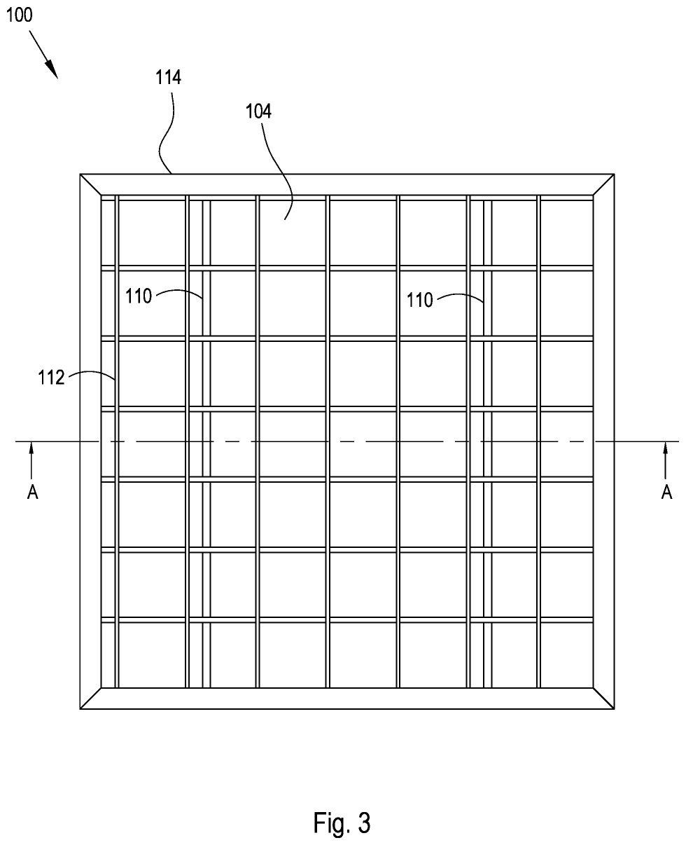

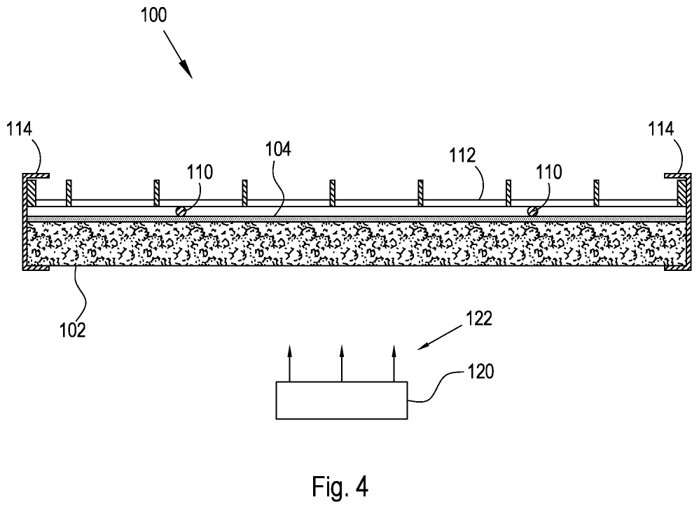

FIGS. 1-4 illustrate a panel 100 for an air circulation system. Some details are most apparent from FIG. 4 (the cross-sectional view). The panel 100 has a foam layer 102 with a graphite coating 104 provided on one side of the foam layer 102. More specifically, the foam layer 102 is dielectric-type open pore foam made of polyurethane with or without charcoal filling. The foam layer 102 is about 25 mm thick.

The foam layer 102 is coated on one side (one side only, in this example) with a graphite coating 104. The coating 104 is produced by the following procedure:
Providing a natural graphite powder having a particle size of 7-9 microns.
Mixing the graphite powder with phosphoric acid and a bonding agent of an acrylic or polyurethane type in the following proportions (by weight): 70% graphite powder, 10% phosphoric acid, and 20% bonding agent.
Mixing for a duration of 5-10 minutes continuously, to ensure that the graphite is exfoliated into amorphous particle fullerene graphene structures (which may be flakes or nanotubes).
Spraying the mixture with an airless type spray gun onto one side of the foam layer 102 for a thickness of 1-2 mm, such that it is deposited onto a surface of the foam layer 102 and does not penetrate the foam layer 102.
Drying the foam layer 102 with the sprayed mixture in a suitable oven at approximately 60° C. until dry, thereby to form the graphite coating 104.

This process may ensure that one side of the panel 100 is electrified or charged evenly to ensure conductivity through an entire surface of the panel 100.

A pair of metal, e.g., copper or brass, electrodes 110 are provided in electrical contact with the graphite coating 104 adjacent opposite sides of the panel 100 and orientated parallel and transversely spaced apart from each other. The electrodes 110 may be operated to electrify the graphite coating 104 such that the electrification may be tested at any place on the graphite coating 104.

While the electrodes 110 may be rigid and provide a degree of support, the panel 100 includes a support structure 112, 114 in the form of a support layer 112 and a support frame 114. The support layer 112 is in the form of a plastic grid 112 comprising orthogonal members resembling an egg crate. The plastic grid 112 is rigid compared to the foam layer 102 and resists bending and twisting. The plastic grid 122 could have been affixed directly to the graphite coating but in this example is attached (e.g., adhered) to the electrodes 110.

Further, the support frame 114 is of metal and extends around a periphery of the foam and support layers 102, 112. Sides of the foam and support layers 102, 112 may be adhered to the support frame 114 or merely clamped therein. The support frame 114 effectively sandwiches the graphite coating 104 between the foam layer 102 and the support layer 112 with the electrodes 110 provided between the graphite coating 104 and the support layer 112. (Attachment formations (not illustrated) may be provided on the support layer 112 and/or the support frame 114.)

An ioniser 120 is provided as part of the panel 100. The ioniser 120 may be integrated with the panel 100, e.g., affixed to the support frame 114, or separate, but mountable to, or adjacent, the foam layer 102. The ioniser 120 is mounted about 5 mm below the foam layer 102 and directed towards the foam layer 102.

In use, a side with the support structure 112 visible (with the graphite layer 104 underneath) is intended to be the front-facing side; in other words, the side which air is to be circulated past. It will be noted that air is not circulated through the foam layer 102 or the graphite coating 104 and the panel 100 is therefore not a filter in the conventional sense. The foam layer 102, with the adjacent ioniser 120, is a back-facing side of the panel 100.

The ioniser 120, in use, is directed towards the foam layer 102 to emit ionised particles 122 towards the foam layer 102. These ionised and charged particles 122 pass through the foam layer 102 and interact with the graphite coating 104 to charge the graphite coating with an electrostatic charge. This charged graphite coating 104 serves to attract contaminants electrostatically, without air actually having to pass through the graphite coating 104. The support layer 112 may, if desired, be shaped and configured to help direct airflow. The graphite coating 104 has anti-microbial properties thus serving to deactivate or sanitise the contaminants. As a further measure, the electrodes 110 may be energised to pass a current through the graphite coating 104 to further destroy or sanitise contaminants extracted from the air passing the panel 100.

The panel 100 may be installed in a building, for example, adjacent air-conditioning inlets or outlets. Differently shaped, e.g., smaller, variants of the panel 100 may be installed in vacuum cleaners or air purifiers, e.g., instead of, or in addition to, HEPA filters. In fact, the panel 100 could be placed at any location that air passes, even buildings without an air-conditioning system but with some degree of airflow.

Without being bound by theory, the Inventor speculates that the panel 100 can be used to kill bacteria and viruses in indoor environments, to ensure pathogen-free air. The panel 100 could be used alone, or in addition to other air filtration apparatus, to promote cleaner, healthier air.

At least one advantage of the present panel 100 over a more conventional filter is that filter can be clogged, leading to blockages. As the panel 100 is not a filter, it will not get clogged completely and not affect the performance; it can be cleaned by vacuuming or other methods.

What is claimed is:

1. A panel for an air circulation system, the panel comprising:
a foam layer comprising open pore foam;
a support structure configured to support the foam layer;
a graphite coating provided on at least one side of the foam layer; and
an ioniser provided adjacent the foam layer, the ioniser configured to ionise particles thereby to apply a static charge to the foam layer,
wherein the charged foam layer is configured, in use, to attract and trap contaminants from air.

2. The panel as claimed in claim 1, in which the foam layer is of dielectric-type foam made of polyurethane.

3. The panel as claimed in claim 1, in which the foam layer is 20-30 mm thick.

4. The panel as claimed in claim 1, in which the support structure includes a support layer provided parallel to the foam layer.

5. The panel as claimed in claim 4, in which the support layer is of a rigid polymer and include a grid, mesh or web of support structures.

6. The panel as claimed in claim 4, in which the graphite coating is provided between the foam layer and the support structure.

7. The panel as claimed in claim 1, in which the support structure comprises a frame provided around sides of the foam layer.

8. The panel as claimed in claim 1, in which the graphite coating comprises graphene or graphite powder.

9. The panel as claimed in claim 8, in which the graphite powder is 7-9 microns in diameter.

10. The panel as claimed in claim 8, in which the graphite coating comprises 70% (by weight) graphite powder.

11. The panel as claimed in claim 1, in which the graphite coating comprises an acid.

12. The panel as claimed in claim 11, in which the acid is phosphoric acid and the graphite coating comprises 10% (by weight) phosphoric acid.

13. The panel as claimed in claim 1, in which the graphite coating comprises a bonding agent.

14. The panel as claimed in claim 13, in which the graphite coating comprises 20% (by weight) bonding agent.

15. The panel as claimed in claim 1, in which the graphite coating is 1-2 mm thick.

16. The panel as claimed in claim 1, comprising attachment formations for attaching the panel to a base structure.

17. The panel as claimed in claim 1, in which the ioniser is an electronic ioniser configured to emit 1-106 ionised particles.

18. The panel as claimed in claim 1, in which the ioniser is spaced 5 mm from the foam layer.

19. The panel as claimed in claim 1, comprising electrodes sandwiched between the support structure and the graphite coating.

20. An air circulation system which comprises the panel as claimed in claim 1.

21. A method of manufacturing a panel for an air circulation system, the panel comprising a foam layer comprising open pore foam, a support structure configured to support the foam layer, a graphite coating provided on at least one side of the foam layer, and an ioniser provided adjacent the foam layer, wherein the ioniser is configured to ionise particles thereby to apply a static charge to the foam layer and wherein the charged foam layer is configured, in use, to attract and trap contaminants from air, the method comprising:
mixing graphite powder with an acid and a bonding agent;
stirring the mixture to exfoliate it into amorphous particle fullerene graphene structures; and
applying the mixture to the foam layer.

22. The method as claimed in claim 21, in which the stirring is for 5-10 minutes and is continuous.

23. The method as claimed in claim 21, in in which the applying comprises spraying with an airless-type spray gun.

24. The method as claimed in claim 21, which comprises drying the panel in an oven or similar heat applicator at 60° C.
